(19) United States
(12) Patent Application Publication
(10) Pub. No.: US 2020/0129132 A1
(43) Pub. Date: Apr. 30, 2020

(54) Alarm based sleep management system

(71) Applicant: Michael Daniel Coleman, Round Rock, TX (US)

(72) Inventor: Michael Daniel Coleman, Round Rock, TX (US)

(21) Appl. No.: 16/172,737

(22) Filed: Oct. 27, 2018

(51) Int. Cl. A61B 5/00; H04L 29/08; A61B 5/11; A61B 5/01

(52) U.S. Cl. CPC A61B 5/746; H04L 67/12; A61B 5/1113; A61B 2562/0219; A61B 5/4809; A61B 5/1115; A61B 5/01

(57) ABSTRACT

A monitoring system for sleep, awake, vegetative or semi-comatose states, and transmitter for the sensor data monitored to a receiving system for data storage and analysis where that analysis can respond with an alarm or log for future analysis by health care professional or caregiver.

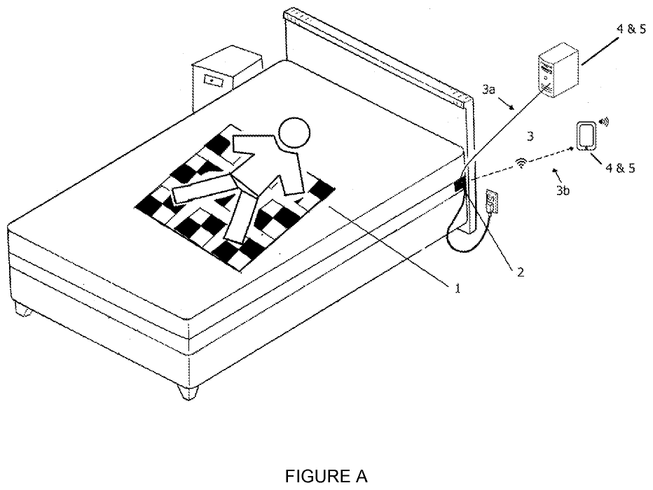

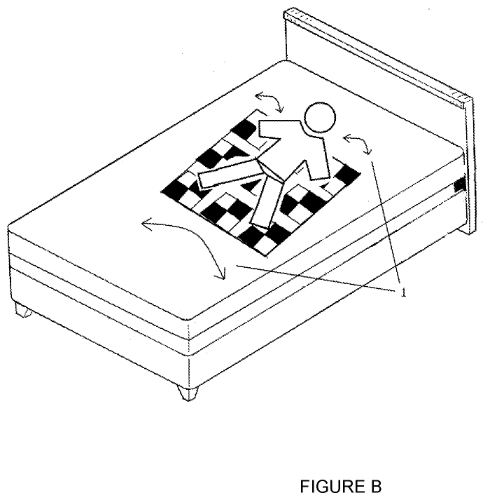

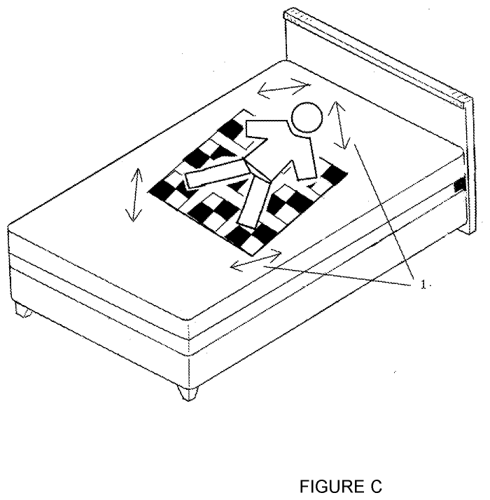

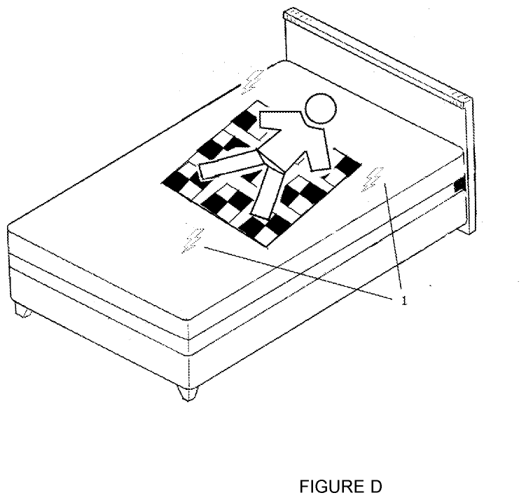

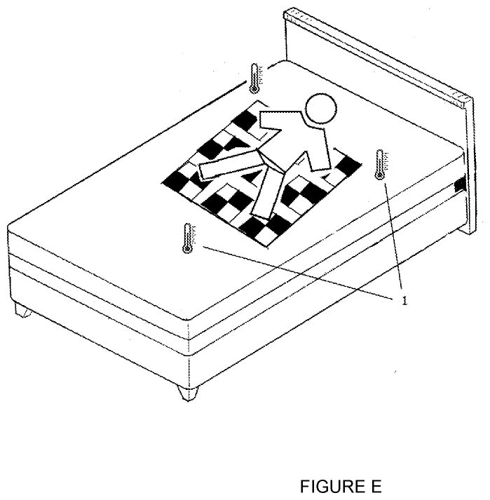

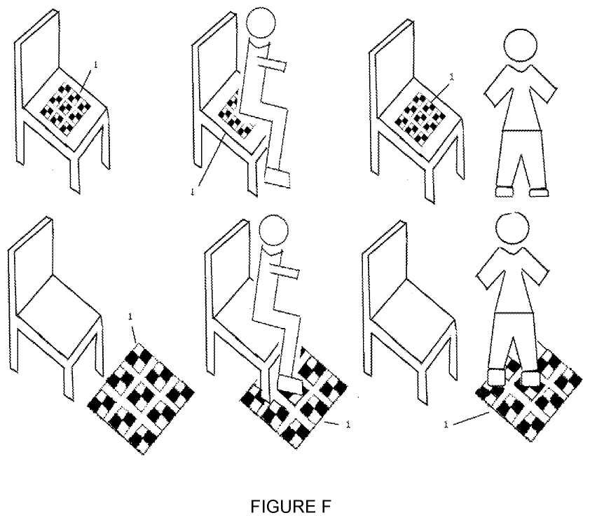

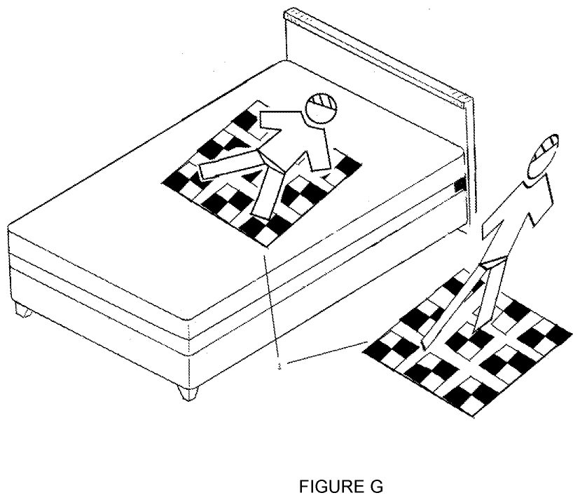

U.S Patent Documents

| | | |
|---|---|---|
| 5,764,153 | Jun. 9, 1998 | Vedaa |
| 6,072,384 | Jun. 6, 2000 | Baker |
| 2008/0191885 | Aug. 14, 2008 | Loree & Toops |
| 2011/0085423 | Apr. 14, 2011 | Cottrell |
| 2011/0125063 | May 26, 2011 | Shalon & Shalon |
| 2011/0308015 | Dec. 22, 2011 | Newham |
| 2012/0053424 | Mar. 1, 2012 | Kenalty, Gordon, & Katz |
| 8,634.279 | Jan. 21, 2014 | Du, Zwartkruis-Pelgrim, & Aliakseyeu, |
| 8,760,976 | Jun. 24, 2014 | Kim |
| 9.271,665 | Mar. 1,2016 | Sarrafzadeh, Xu, Huang, Raut, & |
| | | Yadegar |
| 2016/0217662 | Jul. 28, 2016 | Kashar |
| D762,079 | Jul. 26, 2016 | Oberwelz |

### RELATED APPLICATIONS

This application claims the benefit of provisional patent application No. 62/578,414 to Michael Daniel Coleman et al., filed Oct. 28, 2017, titled “Alarm Based Sleep Management System”, which is incorporated by reference herein.

### FIELD OF THE INVENTION

The present invention relates to sleep, awake, vegetative or semi-comatose state monitoring, particularly, the information data monitored can activate a receiving system to respond to or log said notification.

### BACKGROUND OF THE INVENTION

In the managed care environment, such as an in-home out patient care, nursing home, extended care or hospitals to name a few, the management of sleep has generally been logged or observed by visual process or mechanical devices. This data collection can be done by the patient themselves (if they are capable) but in most cases is done by a caregiver or medical professional and in some cases by a family member or friend. The accuracy of these systems of management have many flaws, such as the inaccurate information entered or monitored by an individual, or the frequency or unavailability of an individual providing the management on an exacting schedule.

In the more recent past this logging and monitoring has been through localized data entry to a hand held smart phone or tablet entry device, this method increases accuracy but is still dependent on the individual's availability. In last few years the monitoring or logging has been achieved through wrist attached devices such as smart watches, and exercise/health trackers. Although these newer wrist attached deices provide monitoring or logging that can accurately log data and time, the input data is limited to information received specifically around or from the wrists proximity, therefore are unable to collect sensor data from specific areas required such as an example, leg movement associated with restless leg syndrome.

The need to have accurate and timely, sometimes time critical data logging, reporting and response is why an alarm based sleep management system is a highly valued system today.

### SUMMARY OF THE INVENTION

The intended outcome benefits of this alarm based sleep management system invention are to monitor a patient who may be incapable or have limited means of data logging, reporting and response.

The present invention relates to a normal sleep state (normal being defined as an individual who is not incapable or has a limited process physical or mental capability to monitor and log their sleep wake cycles and activity). This sleep management system will log, report and notify different states of sleep and its movement or lack of movement.

The invention is able to be used as a fall monitor. As we see healthcare is getting more expensive, a device like this will be useful for families who can't afford to put a family member in a nursing home. But will allow the caregiver or family member to put the device on the user's bed, chair or wheelchair, sofa, floor to monitor for at risk for falls, seizure, dementia/Alzheimer's or elderly living alone.

Although there is no transfer of the patients personal information which could compromise patient confidentiality, a patient identification and security can be achieved through a serial number embedded in the solid state movement detection circuit and that serial number being only known to the data receiver.

The system may have temperature and pressure monitors which may generate alarms based upon unexpected conditions, such as weight (with or without human sleep form sensing) absent of temperature, which may indicate a hypothermic condition requiring assistance; temperature absent weight, which may indicate a situation where a patient may be febrile or where a heating pad or blanket has been left on, and the like.

The invention is able to be used as a vegetative or semi-comatose state, where a time variable can be set as a function of a patient being in a vegetative or semi-comatose state and requiring the movement of the patient to prevent bed sores, the system would send an alarm if the system had sensed no activity from a sensor.

In another example it could indicate that the patient was not on the proper sleep schedule and can log and report activity, inactivity, length of and location of body activity, and store and report this data for future analysis by a professional.

The system can monitor movement out of bed and then movement out or the sleeping area activating a possible alarm situation where the patient is not supposed to move without supervision or assistance.

The alarm based sleep management system can track and monitor activity and movement during sleep cycles based on durations of time. It can also be used to track and store activity to the data receiver so as the data can be analyzed by a sleep or medical professional at a future date. This data can monitor the number of times a patient gets up through the night to use the restroom, which might indicate a urinary tract infection or prostrate problem or other medical syndromes.

### BRIEF DESCRIPTION OF THE DRAWINGS

FIG. A is an overview of a typical alarm based sleep management system

FIG. B is an example of a gyroscope sensing type sensor

FIG. C is an example of a accelerometer sensing type sensor

FIG. D is an example of a capacitive touch sensing type sensor

FIG. E is an example of a temperature sensing type sensor

FIG. F is an example of a gyroscope sensing type sensor

FIG. G is an example of a analyses the timing of activity movement over a period of time sensing system

### DETAILED DESCRIPTION OF THE INVENTION

Since an alarm based sleep management system is not standard or common in patient health and safety today the following embodiments of this invention may require a fundamental shift in how the managed care environment incorporates this system.

In some embodiments, the alarm based sleep management system may include as indicated in drawing FIG. A a single movement sensing underlayment (1) FIG. A between the patient and the sleeping surface, where the sensor is connected to a solid state movement detection circuit (2) FIG. A that could be attached to the sensor directly or could be connected through a wire or cable. When the movement detection circuit (2) FIG. A senses movement it then sends data through communications network (3) FIG. A, where such communications network could be connected through a wire or cable (3a) FIG. A, or could connected through a wireless communications path (3b) FIG. A to the detection circuit's information data receiver (4) FIG. A. This detection circuit's information data receiver (4) FIG. A will then store the data received into a log for future analysis (5) FIG. A or will receive detection circuits information data and respond to the information with an alarm (5) FIG. A, or will receive detection circuits information data, analysis it compared to previously stored data and respond to the analyzed information with an alarm (5) FIG. A if the analysis requires an alarm be sent. This is a typical application of the invention.

In another embodiment, the typical application of the alarm based sleep management system may use a movement sensor (1) FIG. A of a pressure sensing type, this type of sensor may simply sense the singular action of pressure on or off, or it may sense a variable action of the sensor in a scale of 0% to 100% pressure.

In other embodiments, the typical application of the alarm based sleep management system may use a movement sensor (1) FIG. B of a gyroscope sensing type, this type of sensor may simply sense the singular action of slight angle of the patient being changed, or it may sense a variable angle actions of the sensor in a scale of 0 to 360 degrees.

In yet another embodiments, the typical application of the alarm based sleep management system may use a movement sensor (1) FIG. C of a accelerometer sensing type, this type of sensor may simply sense the singular action of slight motion of the patient, or it may sense a variable speed of motion actions of the sensor in a scale of 0% to 100% speed.

In some embodiments, the typical application of the alarm based sleep management system may use a movement sensor (1) FIG. D of a capacitive touch sensing type, this type of sensor may simply sense the singular action of static conductance of the patient being present or not, or it may sense a variable static conductance of the patient in a scale of 0% to 100% defined by electronic formula.

In another embodiments, the typical application of the alarm based sleep management system may use a movement sensor (1) FIG. E of a temperature sensing type, this type of sensor may simply sense the singular action of body temperature of the patient being present or not, or it may sense a variable temperature of the patient in a scale of 0% to 100% defined by electronic formula which might indicate an additional variable of the health of the patient.

In yet another embodiment, the typical application of the alarm based sleep management system may use multiple sensors in combination as an example: a pressure sensing type, this type of sensor could sense the presence of a patient and could then activate a accelerometer sensing type, this sensor would then sense motion of the patient, this combination could then transmit multiple data sets through the system to the detection circuit's information data receiver.

In other embodiments, the alarm based sleep management system may integrate a control feature where the detection circuit's information data analyses the lack of movement over a period of time. This time variable can be set as a function of a patient being in a vegetative or semi-comatose state and requiring the movement of the patient to prevent bed sores, the system would send an alarm if the system had sensed no activity from a sensor,.as an example. In another example it could indicate that the patient was not on the proper sleep schedule.

In another embodiment, the alarm based sleep management system may integrate a control feature where the detection circuit's information data analyses the timing of movement over a period of time. This activity movement can be used to track movement cycles (1) FIG. F based on duration of time. It can also be used to track and store activity to the detection circuit's information data receiver so as the data can be analyzed by a sleep or medical professional at a future date. This data can monitor the number of times a patient gets up through the night to use the restroom, which could indicate a urinary tract infection or prostrate problem. FIG. F indicates an example of a wheelchair situation for patient monitoring.

In some embodiments, the alarm based sleep management system that may contain two or greater (plurality) sensing types that are located on different planes (1) FIG. G, such as the sleeping platform as one plane and the floor as another plane. This could activate a control feature where the detection circuit's information data received from both planes could indicate a fall from the bed to the floor which would activate a alarm notification. Or as another example it could indicate movement out of bed and then movement out or the sleeping area activating a possible alarm situation where the patient is not supposed to move without supervision or assistance, or just store the data for patient activity tracking.

What is claimed is:

1. A Alarm based sleep management system, comprising:
a body movement detection sensor;
a solid state movement detection circuit, with input from movement detection sensor or multiple sensors;
a process of connecting a solid state movement detection circuit to a communications network; and
a process of dynamically controlling said detection circuits information data received over the communications network, such that said information data can activate a receiving system to respond or log said notification.

2. The method of claim 1, wherein said body movement detection sensor or multiple sensors is of a pressure sensing type.

3. The method of claim 1, wherein said body movement detection sensor or multiple sensors is of a gyroscope type.

4. The method of claim 1, wherein said body movement detection sensor or multiple sensors is of an accelerometer type.

5. The method of claim 1, wherein said body movement detection sensor or multiple sensors is of a capacitive touch type.

6. The method of claim 1, wherein said body movement detection sensor or multiple sensors is of a temperature type.

7. The method of claim 1, wherein said body movement detection sensor or multiple sensors is a combination of one or more of type sensors as described in claim 2 through 6.

8. The method of claim 1, wherein analyzing information data activates a receiving system to respond to a lack of movement sensing from said solid state movement detection circuit.

9. The method of claim 1, wherein analyzing information data activates a receiving system to respond to movement sensing from said solid state movement detection circuit.

10. The method of claim 1, wherein analyzed information data from a receiving system logs data for future analysis of movement sensing from said solid state movement detection circuit, where such future analysis activates a response or reporting of resulting analysis.

11. The method of claim 1, further comprising:
a body movement detection sensor;
a secondary body movement detection sensor or multiple sensors;
a solid state movement detection circuit, with input from movement detection sensor and secondary body movement detection sensor or multiple sensors;
a process of connecting a solid state movement detection circuit to a communications network; and
a process of dynamically controlling said detection circuits information data received over the communications network, such that said information data can activate a receiving system to respond or log said notification.

11. The method of claim 1, further comprising:
a body movement detection sensor or multiple sensors on a single plane;
a secondary body movement detection sensor or multiple sensors located on a separate plane from primary body movement detection sensor or multiple sensors;
a solid state movement detection circuit, with input from movement detection sensor and secondary body movement detection sensor or multiple sensors;
a process of connecting a solid state movement detection circuit to a communications network; and
a process of dynamically controlling said detection circuits information data received over the communications network, such that said information data can activate a receiving system to respond or log said notification.
